(19) United States
(12) Patent Application Publication
(10) Pub. No.: US 2010/0331722 A1
(43) Pub. Date: Dec. 30, 2010

(54) Device and method for assessing operant facial pain

(76) Inventors: Robert Martin Caudle, Gainesville, FL (US); John Kim Neubert, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/201,452

(22) Filed: Aug. 10, 2005

Related U.S. Application Data
(60) Provisional application No. 60/600,669, filed on Aug. 10, 2004.

(51) Int. Cl. A61D 99/00; A01K 1/03; A01K 29/00

(52) U.S. Cl. 600/555; 119/421; 119/712; 600/557

(57) ABSTRACT

The subject invention concerns a method and device for assessing facial pain sensitivity exhibited by an animal. The device and method can be used, for example, to evaluate the effect of a disease state, drug, or other intervention, on facial pain sensitivity, such as orofacial pain sensitivity. In one embodiment, the device and method provide a way of assessing both heat and cold sensitivity (hyperalgesia and allodynia) in the facial region in a non-invasive manner. Additionally, since the animals can be kept unrestrained, there are less confounding factors such as stress, which are inherent to other facial pain testing techniques.

### CROSS-REFERENCE TO RELATED APPLICATION

The present application claims benefit of U.S. Provisional Application Ser. No. 60/600,669, filed Aug. 10, 2004, which is hereby incorporated by reference herein in its entirety, including any figures, tables, nucleic acid sequences, amino acid sequences, and drawings.

### BACKGROUND OF THE INVENTION

Uncontrolled pain remains an epidemic public health problem, with a significant portion of this global problem being represented by orofacial pain disorders (e.g., temporomandibular disorders, trigeminal neuralgia, and headaches). These disorders may present with thermal and mechanical allodynia and hyperalgesia. For example, people suffering from trigeminal neuralgia may have severe lancinating pain triggered by an innocuous puff of air on a trigger zone. The characteristics of these clinical disorders are well described; however, evaluation of orofacial pain in animals has proved to be challenging. Previous investigators have adopted various methods intended to produce tonic pain in the orofacial region, including inflammation (Clavelou, P. et al. Pain, 1995, 62:295-301; Haas, D. A. et al. Arch Oral Biol, 1992, 37:417-422; Imbe, H. et al. Cells Tissues Organs, 2001, 169:238-247; Limmroth, V. et al. Pain, 2001, 92:101-106; Pelissier, T. et al. Pain, 2002, 96:81-87; Vos, B. P. et al. J. Neurosci., 1994, 14:2708-2723; and Zhou, Q. et al. J. Comput Neurol, 1999, 412:276-291), neurogenic inflammation (Pelissier, T. et al. Pain, 2002, 96:81-87), and nerve constriction injury (Vos, B. P. et al. J. Neurosci., 1994, 14:2708-2723). However, assessment of trigeminal nerve-mediated nociceptive responses has been limited to a handful of methods that assess processing within the brain stem (e.g., withdrawal responses or grooming) (Clavelou, P. et al. Pain, 1995, 62:295-301; Imbe, H. et al. Cells Tissues Organs, 2001, 169:238-247; Pelissier, T. et al. Pain, 2002, 96:81-87; and Vos, B. P. et al. J. Neurosci., 1994, 14:2708-2723). These unlearned behaviors were elicited by mechanical sensitivity using von Frey filaments (Vos, B. P. et al. J. Neurosci., 1994, 14:2708-2723) or thermal stimulation (Imamura, Y. et al. Exp Brain Res, 1997, 116:97-103). These non-operant assessments are relatively easy to complete but evaluate innate behaviors that do not reveal cerebral processing of nociception. Also, under these assay conditions, it is difficult to eliminate factors such as anticipation or stress when an animal is restrained. Additionally, experimenter bias is difficult to avoid when each stimulus is under manual control (Chesler, E. J. et al. Neurosci Biobehav Rev, 2002, 26:907-923). Previous studies have evaluated the influence of the laboratory environment on animal behavior and found that experimenter identity can play an important influence on behavioral outcome measures (Crabbe et al. Science, 1999, 284:1670-1672; Chesler et al. Neurosci. Biobehav. Rev., 2002, 26:907-923). Therefore, development of investigator-independent outcomes becomes an important consideration when evaluating pain behaviors.

The challenge in developing a behavioral model for assessment of orofacial pain lies in the ability to generate mechanical and thermal stimuli that are not experimenter initiated and generate behavior that is indicative of pain intensity after cerebral processing. Operant conflict paradigms establish a behavioral outcome whereby an animal can decide between receiving a reward or escaping an aversive stimulus (Dubner, R. et al. “A behavioral animal model for the study of pain mechanisms in primates” in Weisenberg, M. Tursky, B. Eds., Pain: New Perspectives in Therapy and Research, New York: Plenum Press, 1976, pp. 155-170). Operant conflict paradigms are advantageous over other stimulus-response assays because the animal can then control the amount of nociceptive stimulation it receives during the testing session (Mauderli, A. P. et al. J. Neurosci Methods, 2000, 97:19-29) using behavioral strategies that are learned and depend upon cerebral processing of input from segmental nociceptive pathways. Reward/conflict paradigms involve operant behaviors that allow the animal to choose between receiving a positive reward or escaping an aversive stimulus (Vierck et al. Exp. Brain Res., 1971, 13:140-158; Dubner et al. “A behavioral animal model for the study of pain mechanisms in primates” in: Weisenberg et al. Eds., Pain: New Perspectives in Therapy and Research, Plenum Press, New York, 1976, pp. 155-170), or to choose between escape from a nociceptive stimulus and escape from another aversive stimulus (Vierck et al. Exp. Brain Res., 1971, 13:140-158). Reward/conflict paradigms are particularly useful for comparison to human studies because the animal can choose a response strategy and thus the data achieved are not experimenter derived or driven.

It is evident from the foregoing that orofacial pain has been well-characterized clinically, but evaluation of orofacial pain in animals has not kept pace. It would be advantageous to have available a device and procedures for examining operant facial pain behavior in animals, such as rodents, under varying levels of stimulation. Additionally, it would be useful to have available a model of orofacial inflammatory pain that could be used with and without a standard analgesic, such as morphine. This would be of particular value for evaluating safety and efficacy of new drugs and provide a key step for advancement of translational pain research.

### BRIEF SUMMARY OF THE INVENTION

The subject invention concerns a device for assessing facial pain sensitivity exhibited by an animal. The device can be used, for example, to evaluate the effect of a disease state, drug, or other intervention, on facial pain sensitivity including, but not limited to, orofacial pain sensitivity. The device is designed to follow a reward/conflict paradigm that is particularly useful for comparison to human studies because the animal can choose a response strategy and thus the experimental data is not skewed by the experimenter's influence.

The device comprises a means for providing an aversive stimulus to a test animal; and a means for providing a reward (positive reinforcement) to the test animal, wherein the means for providing the aversive stimulus and the means for providing the reward are spatially arranged with respect to one another that such that the test animal must contact the means for providing an aversive stimulus (or otherwise expose itself to the aversive stimulus) in order to access the reward.

In some embodiments, the aversive stimulus can be an aversive temperature. In such cases, the means for providing the aversive stimulus is preferably a thermode. The thermode can comprise one or more conducting metal tubes. Preferably, the thermode is arranged to be contacted by the test animal when the test animal makes an attempt to access the reward. The aversive temperature may be hot or cold.

If the device is to be used to measure orofacial pain sensitivity in a test animal, the test animal's face should contact the means for providing the aversive stimulus when attempting to access the reward.

In some embodiments, the aversive stimulus is a mechanical stimulus. For example, in such cases, the means for providing the aversive mechanical stimulus can comprise one or more filaments.

In one embodiment, the device comprises two means for providing aversive stimuli, wherein a first means for providing aversive stimuli provides aversive mechanical stimulus and wherein a second means for providing aversive stimulus provides aversive temperature stimulus, such that the test animal must choose between an aversive temperature stimulus and an aversive mechanical stimulus when attempting to access the reward.

Optionally, the device further comprises a computer data acquisition system, which is in operable communication with the means for providing the aversive stimulus, or with the means for providing the reward, or with both. Preferably, the computer data acquisition system collects and records data from the means for providing the aversive stimulus, the means for providing the reward, or from both, and facilitates the determination of one or more pain measures from the test animal. Examples of such pain measures include, but are not limited to, the number of times the test animal accesses the reward; the amount of reward taken; the number of times the test animal contacts the means for providing the aversive stimulus; the ratio of the number of times the test animal accesses the reward to the number of times the test animal contacts the means for providing the aversive stimulus; and the relationship between the duration of contact between the test animal and the means for providing the aversive stimulus and the number of times the test animal contacts the means for providing the aversive stimulus.

The subject invention also pertains to a method for testing pain sensitivity exhibited by a test animal, using the device of the invention. The device and method of the invention can be of significant aid in screening of drugs or compounds targeted at reducing pain in the facial region, such as compounds to treat headaches. The method of the invention comprises introducing the test animal to the device and determining at least one pain measure from the test animal. Preferably, the pain measure includes at least one selected from among the number of times the test animal accesses the reward; the amount of reward taken; the number of times the test animal contacts the means for providing the aversive stimulus; the ratio of the number of times the test animal accesses the reward to the number of times the test animal contacts the means for providing the aversive stimulus; and the relationship between the duration of contact between the test animal and the means for providing the aversive stimulus and the number of times the test animal contacts the means for providing the aversive stimulus.

### BRIEF DESCRIPTION OF THE DRAWINGS

FIG. 1 shows selective facial hair removal for specific trigeminal nerve division testing. The dotted region outlines the typical shaving pattern of the face. The metal tubing of the thermode can be positioned such that different trigeminal nerve dermatomes (e.g., maxillary or mandibular) of the shaved region are in contact.

FIGS. 2A and 2B show a schematic diagram (FIG. 2A) and a photograph (FIG. 2B) of the operant thermal testing device. In a typical testing session, the animal will access the reward bottle by first touching the thermode. The online trace recordings (Contacts, Licks) provide immediate feedback to the investigator to ensure the animal is being stimulated by the thermode each time it accesses the reward (FIG. 2A). The animals' face protrudes through the thermode opening such that the whiskers are not touching the thermode, as seen in the photograph of the actual testing device (FIG. 2B).

FIG. 3 shows acquisition of contact attempts and licking episodes at 37.7° C. These traces (facial stimulus contacts, reward bottle licking contacts) represent a typical recording for an animal accessing the milk bottle during the standard 30-minute testing session. Note that a facial contact recording can occur without a bottle lick recording (denoted by the arrows), indicating an unsuccessful attempt for receiving the reward in the presence of the stimulus.

FIGS. 4A-4F show the effects of testing order on operant outcome measures. In a subset of animals (N=8), the six outcome measures (intake, FIG. 4A; facial contact duration, FIG. 4B; licking contacts, FIG. 4C; facial contacts, FIG. 4D; ratio licks/contact events, FIG. 4E; and ratio facial duration/facial contacts, FIG. 4F) were calculated and plotted based on a fixed, non-sequential testing order. These data show that the testing order did not affect the results. The arrows (FIGS. 4C and 4D) denote the general direction of increasing stimulus temperature and note that the cumulative number of events is displayed over the course of the experiment.

FIGS. 5A-4F show the effects of temperature on operant outcome measures. The animals displayed an aversive behavior to the higher temperature stimuli, as noted by a significant decrease in mean reward solution intake (FIG. 5A) and mean total facial contact duration (FIG. 5B). Increasing the stimulus temperature significantly reduced the number of mean successful licks (FIG. 5C) while increasing the number of mean attempts (FIG. 5D). Lastly, the pain index ratios of reward/attempts (FIG. 5E) and facial duration/contacts (FIG. 5F) were also significantly reduced as thermode temperature increased. *denotes a significantly (P<0.05) lower value and +denotes a significantly higher (P<0.05) value compared to all other temperatures in post-hoc analyses.

FIGS. 6A-6F show the effects of inflammation and morphine on operant outcome measures. Carrageenan produced a significant reduction (*P<0.05) in all outcome measures (FIGS. 6A, 6B, 6C, 6E, and 6F) at 45.5° C. with the exception of facial contact events (FIG. 6D). The hyperalgesic effect of inflammation was completely blocked when animals were pretreated with morphine (0.5 mg/kg, s.c.) 30 minutes prior to testing. Facial contact events were significantly higher (*P<0.05) for uninflamed animals tested at 45.5° C. compared to the other test groups.

FIGS. 7A and 7B show post-inflammatory operant responses prior to and after morphine administration. For a single animal, repeated testing following carrageenan administration was used to evaluate whether morphine could reverse the inflammatory hyperalgesia. The time course of this experiment is illustrated in (FIG. 7A). As seen in the upper and lower left traces, the animal made many facial attempts, but had few reward successes. There was a dramatic increase in the number of reward successes (lower right trace) following morphine administration (FIG. 7B). The number of facial contacts decreased following morphine (upper right), but the duration increased, and in turn the two pain indices (ratio licks/contact events and ratio facial duration/facial contact) both increased, indicating an analgesic response induced by morphine.

FIGS. 8A and 8B show schematic diagrams of an embodiment of the operant orofacial pain assessment device of the present invention. FIG. 8A shows a means for providing an aversive temperature stimulus (thermode) behind a means for providing a mechanical aversive stimulus (bristles).

### DETAILED DESCRIPTION OF THE INVENTION

The present invention provides a device and method for measuring pain sensitivity in a test animal, such as a rodent or any other mammal that is trainable on the device and that has pain receptors in the desired anatomical location. Using the method and device of the invention, an investigator can predict or extrapolate human clinical pain, such as orofacial pain.

The device of the invention comprises a means for providing an aversive stimulus to a test animal; and a means for providing a reward (positive reinforcement) to the test animal, wherein the means for providing the aversive stimulus and the means for providing the reward are spatially arranged with respect to one another that such that the test animal must contact the means for providing an aversive stimulus (or otherwise expose itself to the aversive stimulus) in order to access the reward. The method of the invention comprises introducing the test animal to the device and determining at least one pain measure from the test animal. Preferably, the pain measure includes at least one selected from among the number of times the test animal accesses the reward; the amount of reward taken; the number of times the test animal contacts the means for providing the aversive stimulus; the ratio of the number of times the test animal accesses the reward to the number of times the test animal contacts the means for providing the aversive stimulus; and the relationship between the duration of contact between the test animal and the means for providing the aversive stimulus and the number of times the test animal contacts the means for providing the aversive stimulus.

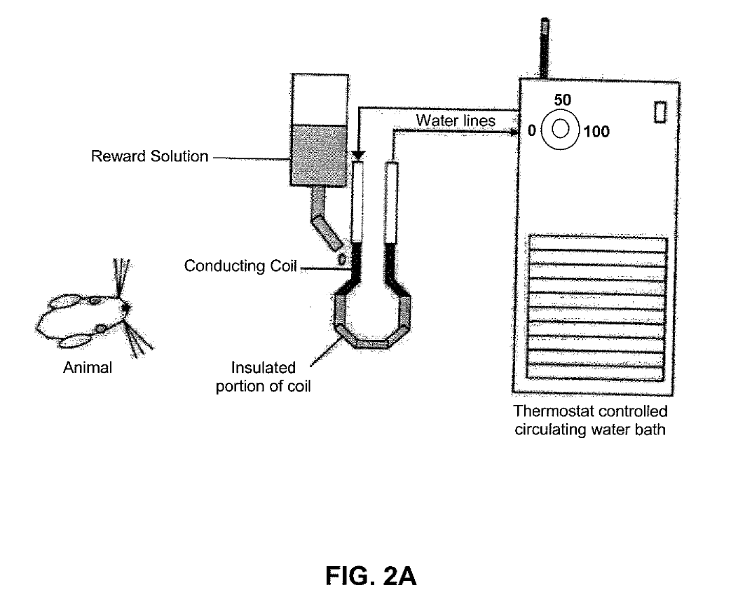

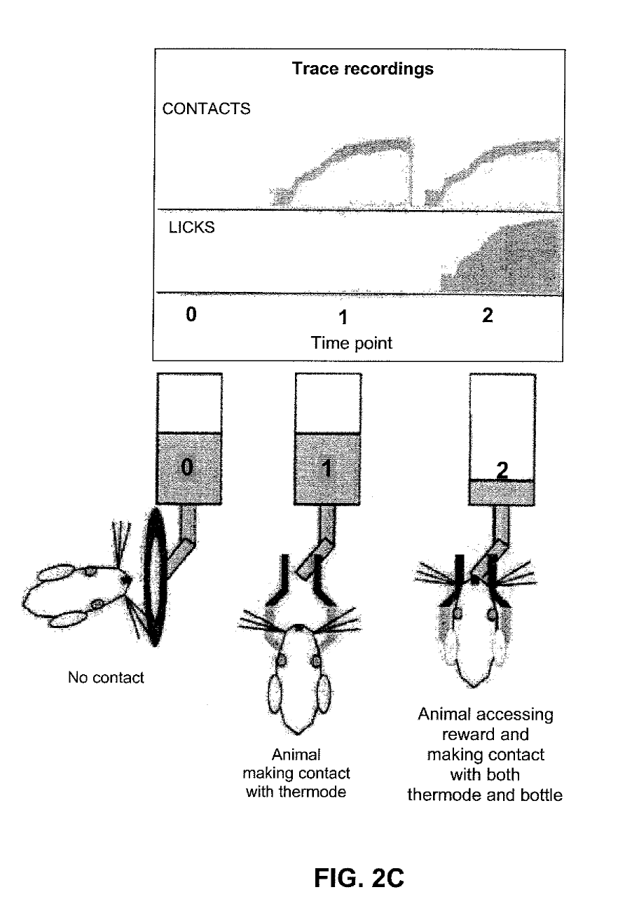

Optionally, the device of the invention further comprises a chamber having a wall with an aperture through which the test animal must extend its head to access the reward, as shown in FIGS. 2A and 2B (wall not shown). In one embodiment, the means for providing the aversive stimulus is outside the chamber and adjacent to the aperture. In another embodiment, the means for providing the aversive stimulus is within the chamber and adjacent to the aperture. In another embodiment, the means for providing the aversive stimulus at least partially lines the aperture.

In one embodiment, the chamber includes two or more apertures, forcing the animal to choose between different types of aversive stimulus, e.g., a mechanical aversive stimulus and a temperature aversive stimulus. In another embodiment, the chamber includes two or more apertures with different types of aversive temperature stimulus, forcing the animal to choose between a hot aversive stimulus and a cold aversive stimulus.

In another embodiment, the device of the invention includes a partition that encloses the means for providing the reward, wherein the partition has an aperture through which the test animal must extend its head to access the reward. The partition can be of constructed of any material (such as plastic, wood, metal, woven or non-woven fabric, or glass) and be of any shape. The partition can be transparent, partially transparent, or opaque. Thus, in this embodiment, the partition effectively prevents the test animal from accessing the reward (i.e., from any practical direction) without going through the aperture. In this embodiment, the device can be placed anywhere within a standard animal testing chamber, including those that lack apertures to accommodate the device of the invention. In this way, the device is modular and can be used in conjunction with any of a variety of animal behavior testing chambers. In one embodiment, the device can include two or more apertures, forcing the animal to choose between different types of aversive stimulus, e.g., a mechanical aversive stimulus and a temperature aversive stimulus. In another embodiment, the device includes two or more apertures with different types of aversive temperature stimulus, forcing the animal to choose between a hot aversive stimulus and a cold aversive stimulus. In this embodiment, a single reward can be shared between the apertures, or separate rewards (which can be the same or different) can be provided. In another embodiment, two different devices of the invention can be placed in a chamber, one device with an aperture providing access to a reward and requiring exposure of the animal to a hot aversive stimulus, and another device of the invention with an aperture providing access to a reward and requiring exposure of the animal to a cold aversive stimulus. Again, both devices include a partition forcing the animal to place its head through the apertures if the animal is to access the reward.

The aperture of the device can be any shape (i.e., irregular, circular, etc.). When the device and method of the invention are used for measuring orofacial pain sensitivity, the aperture is large enough such that the animal can place its head through the aperture to access the reward but is preferably not large enough to completely move through the aperture. For example, when the test animal is a rat, the aperture can be 4-5 centimeters wide and 10-15 centimeters tall.

In some embodiments, the aversive stimulus is an aversive temperature. In such cases, the means for providing the aversive stimulus can be a thermode. The thermode can comprise one or more conducting metal tubes. Preferably, the thermode is arranged to be contacted by the test animal when the test animal makes an attempt to access the reward. The aversive temperature may be hot or cold. The temperature may vary somewhat with the particular species of test animal. For example, a temperature within the range of 2° C. to 15° C. or within the range of 45° C. to 70° C. is aversive to many species, such as rodents. Preferably, the hair is removed from the area of contact between the test animal and the means for providing the aversive stimulus. The conducting tubing can be composed of one or more materials such as copper, aluminum, steel, etc. If orofacial pain is to be measured, the conducting tube can be arranged to be contact unilaterally on the face of the animal or bilaterally on the face of the animal when the animal attempts to access the reward. The conducting tube can have any cross-sectional shape. The conducting tube can be a single tube forming a loop or coil (such as shown in FIG. 2A) or two or more separate conducting tubes can be utilized (as shown in FIG. 2B). The conducting tube(s) can be partially insulated. The conducting tubes can be hollow or solid, depending upon the manner in which the tubes provide aversive stimulus. For example, if it is necessary for fluid to pass fluid to heat or cool the tubes for an aversive temperature, the tubes must necessarily be at least partially hollow.

Optionally, the means for providing the aversive temperature stimulus can comprise one or more thermoelectric modules (“TEMs”; also known as Peltier devices) which can be used as heat pumps to move heat to and from the thermodes (see, for example, U.S. Pat. No. 6,637,372 (Mauderli and Vierck), which is incorporated herein by reference in its entirety). A TEM contains a number of p-type and n-type pairs (couples) connected electrically in series and sandwiched between two ceramic plates. When connected to a DC power source, current causes heat to move from one side of each TEM to the other side, creating a hot side and a cold side. If the current is reversed (reversing the polarity of the power supply), the heat is moved in the opposite direction, with the hot face becoming the cold face and vice-versa. TEMs are particularly well suited for use in the device of the subject invention because they are solid-state devices that can provide precision temperatures, with no moving parts. Therefore, they produce virtually no noise to distract the test animal, which could potentially skew experimental results. The amount of heat pumped through the TEM is directly proportional to the power supplied. Temperature can be controlled through manual or automatic means. An automatic temperature controller can be utilized and can range in complexity from a simple on-off thermostat to a complex computer controlled feedback proportional control loop. Each heat sink is preferably a liquid-type heat sink composed of aluminum and containing channels to remove heat from the surface of the TEM that contacts it, or to supply heat to the surface of the TEM that contacts it, depending upon the direction the heat is pumped by the TEM. Other types of heat sinks, such as fin-type heat sinks, with or without fans, can also be utilized. In order to maximize heat transfer between each TEM and each heat sink, and between each TEM and the test animal, a film of heat conducting medium, such as zinc oxide paste, can be applied to the top and bottom surface of the TEM.

The location of the test animal relative to the device can be determined at a given time visually by an operator either directly or indirectly, e.g., directly via eyesight or with a video camera. However, the device of the subject invention can also include means for sensing the presence of the test animal relative to the device and, preferably, relative to the aperture. For example, means for sensing the presence of the test animal can include one or more infrared beam emitters and detectors operably positioned. In those embodiments including a chamber, the emitters and detectors can be disposed within the chamber, for example. As used in this context, the term “operably positioned” means that the emitters and detectors are at a predetermined position with respect to each other such that one or more infrared beams are emitted and detected, and interruption of the infrared beams by the test animal produces a signal (such as electronic, visual, and/or audible) indicating the location of the test animal relative to the device and, preferably, the aperture. The amount of time spent in proximity to the aperture without entering could be determined, for example.

Optionally, the device further comprises a computer data acquisition system, which is in operable communication with the means for providing the aversive stimulus, or with the means for providing the reward, or with both. Computer data acquisitions systems are well known in the art. Preferably, the computer data acquisition system collects and records data from the means for providing the aversive stimulus, the means for providing the reward, or from both, and facilitates the determination of one or more pain measures from the test animal. Examples of such pain measures include, but are not limited to, the number of times the test animal accesses the reward; the amount of reward taken; the number of times the test animal contacts the means for providing the aversive stimulus; the ratio of the number of times the test animal accesses the reward to the number of times the test animal contacts the means for providing the aversive stimulus; and the relationship between the duration of contact between the test animal and the means for providing the aversive stimulus and the number of times the test animal contacts the means for providing the aversive stimulus. As used herein, the term “computer” refers to a computer data acquisition system.

Optionally, wireless telemetry can be utilized to transmit and receive diagnostic information (e.g., biological information such as heart rate, blood pressure, body temperature, concentration of a biological molecule, etc.) concerning the test animal. Wireless telemetry systems include, for example, radio-electric transmission, optical transmission, ultrasound transmission, or other transmission technologies that do not rely on a continuous wire, lead, or cable connection between the test animal and any external equipment.

Preferably, the test animal is not restrained, thereby eliminating the confounding factor of restraint stress, which is known to affect pain sensitivity.

The test animal can be any mammal having nociceptors in the desired anatomical location and which is capable of exhibiting an operant behavioral response that may be observed and/or recorded (such as a licking event or facial contact event). When orofacial pain sensitivity is to be assessed, the test animal should have facial nociceptors (receptors for facial pain stimuli). Preferred animals are those having a physiology sufficiently similar to humans such that they provide relevant correlative data, as an animal model, for the particular treatment being conducted on the animal. Examples of appropriate animals include those of the order rodentia, such as members of the family muridae (e.g., mice, rats, hamsters, voles, lemmings, and gerbils), lagomorpha (e.g., rabbits, pikas, and hares), and caviidae (e.g., guinea pigs), or those of the order insectivora, such as members of the family soricidae (shrews) and talpidae (moles), dogs, cats, and so forth. It is contemplated that other mammals, such as non-human primates (such as chimpanzees, orangutans, gibbons, and marmosets) could be utilized as the test animal.

The device may further comprise a chamber having an aperture through which the animal must place its head in order to gain access to the reward item (e.g., to consume a food such as sweetened condensed milk or other reward solution). Various means for providing the reward item can be used (such as a platter or bottle). Preferably, at least a portion of the reward providing means is sufficiently conductive such that when the animal contacts the conductive portion an electrical circuit is completed. If the reward item is a solution, the reward providing means can be a standard watering bottle having a metal spout, for example.

Preferably, the aperture is at least partially lined (preferably, entirely lined) with the means for providing the aversive stimulus. For example, the aperture can be at least partially lined with a thermode, bristles, or both. Where the reward item is a solution provided by a watering bottle, the water bottle can include a metal spout and be connected to a power supply (such as a DC power supply). Preferably, the watering bottle is connected to the power supply and, in series, to a computer data acquisition system. The bottle is placed such that the animal, when reaching toward it with its face, will contact the means for providing the aversive stimulus (such as metal tubing), as shown in FIGS. 2A and 2B.

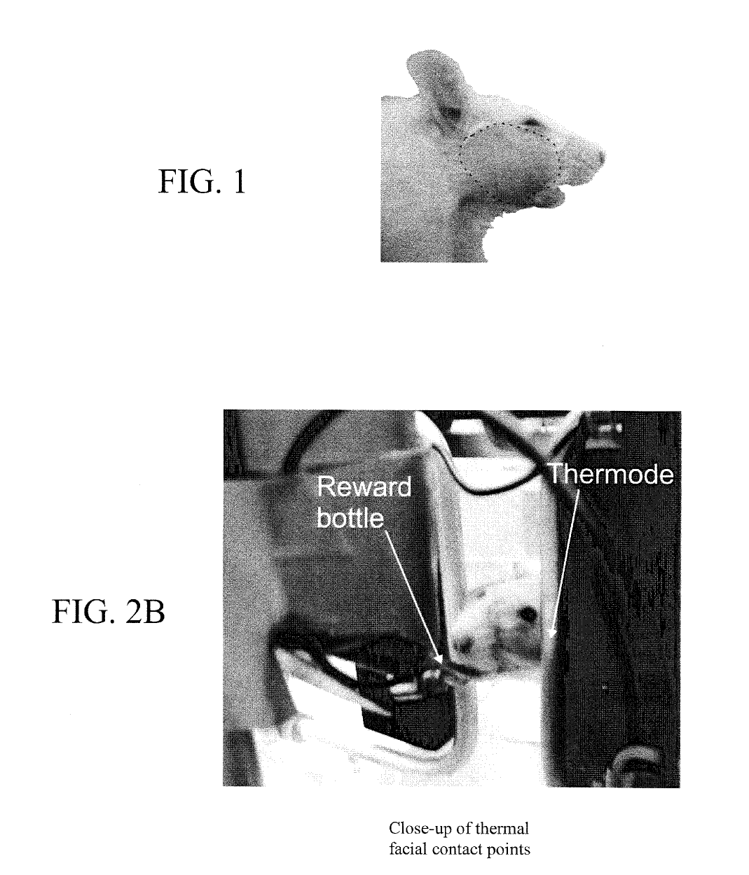

The bottle position can be adjusted horizontally and vertically to target specific areas of the facial region (e.g., maxillary or mandibular divisions). As shown in FIG. 1, regions of the animal's face are preferably shaved to maximize contact. When the animal drinks from the water bottle, the skin on its shaved face will contact the tubing and the animal's tongue will contact the metal spout on the watering bottle, completing the electrical circuit (registered in the computer as a 3 volt step). The animal's face can make contact with the conductive tubing unilaterally or bilaterally.

The device and method of the invention are not limited to assaying orofacial pain sensitivity. For example, the means for providing the aversive stimulus, the means for providing the reward item, the position of the reward item, and/or anatomical regions that are shaved, can each be modified to target other regions of the animal's anatomy (such as other regions of the face, head, neck, feet, etc). For example, the means for providing the aversive stimulus can be a platform or area of floor (base) that provides an aversive temperature (hot or cold) to the test animal when the test animal attempts to access the reward item. Thus, the animal's pain sensitivity at its feet is determined.

The number of times the reward item is accessed, the total number of events, the duration of each rewarding event, and the time course can be observed and, preferably, recorded, e.g., manually by an observer and/or automatically by a computer data acquisition system. For example, if the reward is a consumable fluid such as milk, the frequency of drinking, the total number of events, the duration of the drinking events, and the time course of the drinking can be observed and, preferably, recorded. Additionally, the total amount of reward item consumed or otherwise taken by the animal can be measured and compared between animals and treatments.

Another circuit can be established from the thermode to the animal by grounding the floor with an aluminum sheet, and the same parameters can then be observed and recorded to determine the frequency of contacts with the thermode, the total number of events, and the duration of contacts. This circuit is used to determine if the animal makes an attempt at the reward item but is discouraged by the temperature of the thermode. A complete session will typically last 30 minutes per trial and animals can be tested 3 or 4 times a week. The animals are preferably fasted prior to each testing.

The chamber wall(s) can be constructed of a variety of materials, such as plastic, wood, metal, woven or non-woven fabric, or glass. One or more of the chamber walls can be completely or partially transparent, or opaque. For example, one or more walls can contain a transparent a window. Preferably, the chamber is transparent (e.g., constructed of acrylic or PLEXIGLASS), permitting easy observation of the test animal within the device. In this case, it is also preferable to create a one-way mirror effect by tinting the walls of the chamber and dimming the light in the outside environment to minimize distractions for the test animal. The chamber can be any of a variety of shapes (e.g., square, triangular, circular, irregular, etc.), with the number of walls depending on the shape. For example, if the chamber is circular, it can be considered to have a single wall. Preferably, the chamber has a base for supporting the walls and/or the device's contents. However, the chamber may lack a base and simply be placed on a counter or table top, floor, or other supporting surface during use. Preferably, the chamber has a lid or is otherwise covered to minimize outside stimuli (e.g., from the laboratory environment). The base may be composed of the same materials as the chamber wall(s) or different materials. The lid may be composed of the same materials as the chamber wall(s) or different materials.

A treatment being tested with the device of the subject invention can include administration of a substance (e.g., a drug or nutraceutical), a surgical procedure, and/or other intervention that is being evaluated for its effects on facial pain sensitivity or general effects on operant behavior. Substances to be screened for effects on pain sensitivity (such as analgesic activity) can be administered to the animal by any route (e.g., oral, nasal, or parenteral, such as topical, cutaneous, subcutaneous, intramuscular, etc.) before, during, and/or after experimental trials. The substance can be delivered to the animal by any treatment regimen (for example, by bolus injection or continuous infusion). Although an advantage of the invention is that the test animal is unrestrained, a tether can be utilized to deliver the test substance to the test animal. The substance can be administered via an implant or device, such as an indwelling catheter or infusion device, in a controlled-release fashion, for example. Once a substance is screened and identified as having a desired activity, a pharmaceutical composition can be manufactured, by adding the substance to a pharmaceutically acceptable carrier, for example. A treatment being tested (e.g., a surgical procedure, administration of a test substance, and/or other intervention) can be either hyper-analgesic (decreasing normal pain sensitivity) or hypo-analgesic (increasing pain sensitivity beyond that which is normal). Furthermore, the treatment can be genetic manipulation conducted on either the test animal itself, or one or more of the test animal's forebears. For example, “knock out” animals can be tested with the device of the subject invention to study the effects of the knocked out gene or genes on nociception with or without further treatment. The test animal can be suffering from a disease state or other pathological condition. The pain sensitivity of the test animal suffering from a disease state or pathological condition can be evaluated with the subject device, with or without treatment. Therefore, the device of the subject invention can be used to test the general facial pain sensitivity exhibited by a test animal, in whatever condition the test animal is in, naturally occurring or artificially induced. The device and method of the invention can be used to evaluate pain sensitivity in various injury models (such as inflammation arthritis, and nerve injury) throughout the body. For example, for orofacial injury models, a chemical could be injected into the cheek or mandibular joint of the test animal to model inflammation or arthritis, respectively. For a nerve injury model, a facial nerve could be damaged or tied off to cause a painful syndrome of surgery. Various injury models and outcome measures are known in the art and can be used in conjunction with the device and method of the subject invention (see, for example, Ho, J. et al. J. Pharma. Exp. Therapeu., 1997, 281:136-141; Vierck Jr., C. J. et al. Behav. Neurosci., 2004, 118:627-635; Raboisson, P. and Dallel, R. Neurosci. Biobehav. Rev., 2004, 28:219-226; Benoliel, R. et al. Pain, 2001, 91:111-121; Benoliel, R. et al. Pain, 2002, 99:567-578; and Eliav, E. et al. Pain, 2004, 110:727-737, which are incorporated herein by reference in their entirety).

The device and method of the invention provides an innovative operant behavioral assay with an innovative and sensitive means of detecting and quantifying pain within the facial region and can be completed on several models of facial pain (such as inflammation, arthritis, and nerve injury). The usefulness of the device and method of the present invention derives from the simplicity of the device's design and the wealth of data that can be generated using it. Typical pain assays evaluate rudimentary segments of the pain processing pathway, such as reflex (e.g., limb withdrawal) or unlearned behaviors (e.g., grooming). However, in order to better model the human pain experience, one must consider the effects of higher processing done in the brain. In this reward-conflict assay, this higher level of processing can be assessed whereby the animal must make its own decision on whether it will complete the task based on its pain level. This more closely simulates human pain conditions, as motivation and emotional states influence the experience. Thus, the invention provides an operant assay for evaluating pain within the facial region and provides a pivotal link for translating basic pain research ideas into clinical trial strategies for managing pain.

This non-invasive thermal assessment assay provides a way of assessing both heat and cold sensitivity (hyperalgesia and allodynia) in the facial region. Additionally, since the animals are unrestrained, there are less confounding factors such as stress, which are inherent to other facial pain testing techniques. A significant benefit of the device and method of the invention is that they can be automated once the animal is placed in the chamber; therefore, a high throughput system for behavioral data can be obtained.

The device and method of the present invention is useful for modeling and evaluating pain in the facial region including orofacial pain and craniofacial pain, for example. Thus, this pain includes pain sensation of the intraoral and extraoral structures of the head and face involving the trigeminal, facial, and glossopharyngeal nerves, particularly those sensations carried to the central nervous system (CNS) by the trigeminal system. The trigeminal system refers to the complex arrangement of nerve transmission fibers, interneurons, and synaptic connections which process incoming information from the three divisions of the trigeminal nerve, which contains both sensory and motor fibers (Conti et al., J. Appl. Oral Sci., 2003, 11(1):1-7). Sensory fibers innervate the anterior part of the face, teeth, mucous membranes of the oral and nasal cavities, conjunctiva, dura mater of the brain, and intracranial and extracranial blood vessels. Motor fibers supply the muscles of mastication. Sensory information from the face and mouth (except proprioception) is carried by primary afferent neurons through the trigeminal ganglion to synapse with second order neurons in the trigeminal brain stem complex.

Orofacial pain, like pain elsewhere in the body, is usually, the result of tissue damage and the activation of nociceptors, which transmit a noxious stimulus to the brain (Vickers E. R. and Cousins, M. J., Aust. Endod J., 2000, 2(1):19-26). However, due to the rich innervation of the head, face, and oral structures, causes of orofacial pain are often very complex and difficult to diagnose. Thus, facial pain, as used herein, includes pain caused by, or modeled for, temporal mandibular disorder (TMD) and tension-type headache. The term “TMD” has been used to characterize the generalized nonspecific symptom complex of headache, neck ache, ear pain, face pain, tenderness of muscles to palpation, sensation of bite change, difficulty chewing and/or swallowing, gross joint sounds and limited range of jaw motion.

The following is an outline of the facial nociception assay procedures that may be carried out using the device of the invention and in accordance with the method of the invention. For simplicity, test animal is represented by a rat, the means for providing the aversive stimulus is represented by thermodes, the means for providing the reward to the test animal is a bottle, and the reward (positive reinforcement) is sweetened condensed milk.

### Performance of Facial Nociception Assay and Analysis of Data.

Training:

1. Naive rats are food restricted over night (12-15 hours).
2. The temperature of the thermodes is set to approximately 25° C.
3. The rats are then placed into the testing apparatus for 30 minutes and allowed to explore the environment and drink from the water bottle containing the reward.
4. This procedure is repeated until the rat reaches the criteria of consuming 10 g of sweetened condensed milk solution. Usually, 3 to 6 trials.

Testing:

1. Trained rats are food restricted over night.
2. The temperature of the thermodes is set to the desired testing temperature (2-70° C.).
3. The animals are placed in the testing apparatus and allowed to explore the environment and drink from the water bottle containing the reward.
4. The apparatus is set up so that when the animal contacts the thermodes a circuit is completed and when the animal contacts the drinking bottle a second circuit is completed. Both circuits are monitored through an analog to digital converter and a computer.

Data Analysis:

1. The drinking bottles are weighed before and after testing each animal to determine the amount of sweetened condensed milk that was consumed.
2. The two data sets from the thermode and from the drinking bottle that were collected on the computer are analyzed for number of contacts and duration of each contact.
3. Ratios are calculated by dividing the number of times the animal contacted the drinking bottle by the number of times the animal contacted the thermodes (Reward/Attempts) and by dividing the duration of the thermode contacts by the number of thermode contacts to determine the average duration of contact with the thermode (Facial duration/Contact).

Data that can be presented from this procedure:

1. Reward consumed or otherwise taken by the test animal.
2. Number of thermode contacts.
3. Duration of thermode contacts.
4. Number of drinking bottle contacts.
5. Ratio of drinking bottle contacts to thermode contacts, which represents the amount of effort the animal has to exert to obtain the reward.
6. The average contact duration, which represents the relative tolerance the animal has for the temperature of the thermodes.

The aforementioned data (also referred to herein as outcome or pain measures) are well known in the art (see, for example, Hargreaves K. et al., “A new and sensitive method for measuring thermal nociception in cutaneous hyperalgesia”, Pain, 1988, 32: 77-88; and Eliav E. et al., “The kappa opioid agonist GR89,696 blocks hyperalgesia and allodynia in rat models of peripheral neuritis and neuropathy” Pain, 1999, Feb., 79(2-3):255-64, which are incorporated herein by reference in their entirety.

As used herein, the term “thermode” refers to a device that typically includes a thermoelectric heat pump, a temperature sensor, and a heat sink. The heat pump moves heat into or out the heat sink in order to produce a specific temperature at the surface of the device.

As used in this specification, the singular “a”, “an”, and “the” include plural reference unless the contact dictates otherwise. Thus, for example, a reference to “a chamber” includes more than one such chamber. A reference to “a tubing” includes more than one such tubing. A reference to “an animal” includes more than one such animal.

As used herein, the terms “operable communication” and “operably connected” are used interchangeably and mean that the particular elements are connected in such a way that they cooperate to achieve their intended function or functions. The “connection” or “communication” may be direct or indirect, physical or remote. For example, the means for providing the aversive stimulus and the means for providing the reward are operably connected in that the means for providing the aversive stimulus and the means for providing the reward are spatially arranged with respect to one another such that the test animal must contact the means for providing an aversive stimulus in order to access the reward. The term “operable communication” with respect to the computer data acquisition system means that the computer data acquisition system is linked (directly or indirectly; physically or remotely) with the means for providing the aversive stimulus, or with the means for providing the reward, or with both. Preferably, the link between these components allows passage of data facilitating the determination of one or more pain measures from the test animal, such as the number of times the test animal accesses the reward; the amount of reward taken; the number of times the test animal contacts the means for providing the aversive stimulus; the ratio of the number of times the test animal accesses the reward to the number of times the test animal contacts the means for providing the aversive stimulus; and the relationship between the duration of contact between the test animal and the means for providing the aversive stimulus and the number of times the test animal contacts the means for providing the aversive stimulus.

As used herein, references to “first,” “second,” and the like (e.g., first and second chambers, first and second tubing) are intended to identify a particular feature of which there are at least two. However, these references are not intended to confer any order in time, structural orientation, or sidedness (e.g., left or right) with respect to the particular feature.

The terms “comprising”, “consisting of”, and “consisting essentially of” are defined according to their standard meaning and may be substituted for one another throughout the instant application in order to attach the specific meaning associated with each term.

The terms “hole” and “aperture” are used interchangeably to refer to a space within a chamber wall or partition, through which the test animal must extend its head and/or face to obtain access to the reward item, and thereby expose itself to an aversive stimulus.

The studies described in the Examples provide a thermal operant testing paradigm which tests the hypothesis that animals will display significantly different behaviors as compared to non-painful (control) conditions. The objective of these studies was to describe behavioral responses to facial thermal stimulation and inflammation with/without an analgesic using a novel operant paradigm. Animals were trained to voluntarily place their face against a stimulus thermode (37.7-57.2° C.) providing access to positive reinforcement. These contingencies present a conflict between positive reward and tolerance for nociceptive stimulation. Inflammation was induced and morphine was provided as an analgesic in a subset of animals. Six outcome measures were determined: reward intake, reward licking contacts, stimulus facial contacts, facial contact duration, ratio of reward/stimulus contacts, and ratio of facial contact duration/event. The publication Neubert, J. K. et al. (Pain, 2005, 116:386-395) is incorporated herein by reference in its entirety.

Animals displayed aversive behaviors to the higher temperatures, denoted by a significant decrease in reward intake, total facial contact duration, and reward licking events. The number of facial contacts increased with increasing temperature, replacing long drinking bouts with more frequent short drinks, as reflected by a low ratio of facial contact duration/event. The number of reward licking/facial contact events was significantly decreased as the thermal stimulus intensity increased, providing another pain index derived from this operant method. These outcomes were significantly affected in the direction of increased nociception following inflammation, and these indices of hyperalgesia were reversed with morphine administration. These data reflect an orofacial pain behavior profile that was based on an animal's responses in an operant escape paradigm. This technique allows evaluation of nociceptive processing and modulation throughout the neuraxis. This novel behavioral assessment strategy of orofacial pain provides a key link for completing mechanistic studies that will provide an important advancement in the goal of translational pain research.

### Materials and Methods

General. Male Sprague Dawley rats (200-300 g, N=18) were lightly anesthetized using isoflurane (1-2.5%, inhalation) and their hair was bilaterally removed (FIG. 1) from the orofacial region using clippers, followed by depilatory cream 1 day prior to behavioral testing. Excess cream was removed with a moistened paper towel to minimize skin irritation. Rats were food fasted for 12-15 hours prior to each testing session and following each session were provided with standard food chow. Animals were tested at the same time of the day and a recovery day from the fasting was included between the testing sessions to minimize nutritional differences from their normal food routine. Water was made available ad libitum before and after testing sessions and animal weight was recorded daily. The animals were then brought into the behavioral procedure room 1 hour prior to testing and allowed to acclimate to the temperature and ambient noise of the room. Animal testing procedures and general handling complied with the ethical guidelines and standards established by the Institutional Animal Care and Use Committee at the University of Florida and all procedures complied with the Guide for Care and Use of Laboratory Animals (Council, 1996).

Thermal Testing. A testing cage (20.3 cm W×20.3 cm D×16.2 cm H) with acrylic walls was constructed with an opening in one wall (4×6 cm) which was lined with grounded metal (aluminum) tubing. The tubing served as a thermode when connected to a water pump (Model RTE110B, NES Laboratories, Inc.) via flexible polyethylene tubing through which heated water (range: 37.7-57.2° C.) was circulated (FIGS. 2A and 2B). A standard rodent watering bottle containing a diluted (1:2 with water) sweetened condensed milk solution (Nestle Carnation Company, room temperature) was mounted outside the cage. The circulating water pump was activated and the appropriate temperature was set prior to testing the animals. The room temperature was maintained at 22±1° C. for all behavioral tests.

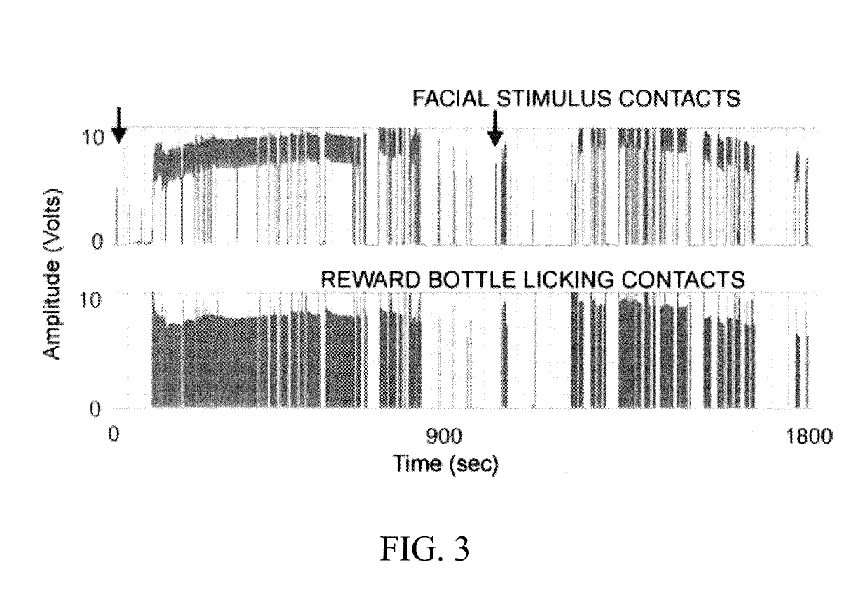

Unrestrained animals were placed separately in a testing cage, and the data acquisition system was activated (WinDaq Lite Data Acq DI-194, DATAQ Instruments, Inc). The bottle was then positioned in proximity to the cage such that the animal was allowed access to the reward bottle when simultaneously contacting the thermode with its face. The metal spout on the watering bottle was connected to a 13-volt DC power supply and, in series, to a multi-channel data acquisition module (WinDaq Lite Data Acq DI-194, DATAQ Instruments, Inc.). When the rat drank from the water bottle, the skin on its shaved face contacted the grounded thermode, and the animal's tongue contacted the metal spout on the water bottle, completing an electrical circuit (FIG. 2A). The bottle position was adjusted horizontally and vertically to facilitate contact of the thermode within the same shaved area of the face for each animal (FIG. 2B). The closed circuit was registered in the computer and data were collected at 60 Hz for the entire length of the experiment. Each spout contact was recorded as a ‘licking’ event. A separate circuit was established from the metal thermode to the animal by grounding the floor with an aluminum sheet for recording of ‘facial contact’ events (FIG. 3). The latter circuit was necessary to determine if the animal made an attempt at the reward bottle but was discouraged by the temperature of the thermode. The duration of each facial contact and the total number of events (licking, facial contact) were recorded. The investigator monitored online data acquisition to ensure that each recorded licking event from the first circuit corresponded to a recorded facial contact on the tubing (the second circuit). This ensured that the animal did not access the reward while avoiding the thermode, and it minimized false-positive recordings of licks.

During offline data analysis, the threshold for detection of the facial contacts and licking contacts was set at 1.0 V, above background noise, to minimize false positive event registration and events typically registered as >5.0 V. An event (licking or facial contact) was registered when the signal went above threshold and ended when the signal dropped below threshold. The cumulative duration and frequency of events were determined for both the licking (reward) contact data and the facial stimulus contact data. A reward/facial contact event ratio was calculated by dividing the number of licking events by the number of facial contact events and the duration per contact for the facial stimulus was also calculated. The total amount of milk consumed (g) was measured and compared at each of the testing temperatures. Data analyses were achieved using custom-written routines in LabView Express (National Instruments Corporation) and Excel (Microsoft).

Animals were first trained to drink milk while contacting the thermode set at 24.3° C. for baseline training (N=5 sessions). This lead-in training period is necessary to acquaint the animals with the task of locating the reward bottle. A subgroup of eight animals was then tested during a 2-week period according to a fixed but non-sequential order of stimulus temperatures (41.7, 37.7, 52.5, 57.5, and 45.5° C.), to assess whether there was an order effect on the outcome measures. A second group of 10 animals was tested using a sequential order of temperatures (37.7-57.2° C.). Stimulus thermode temperatures were verified using a contact thermometer (TC-324B Temperature Controller, Warner Instruments, Inc.). A between group (N=8 vs. 10) analysis was completed at each testing temperature. In addition, a within group comparison (N=10) was completed at the 45.5° C. stimulus for two separate sessions spaced 2 weeks apart.

Orofacial inflammation and analgesia. A model of orofacial inflammatory pain was used as previously described by Ng et al. (Ng and Ong, 2001). Briefly, unanesthetized animals (N=6) were gently restrained and carrageenan (6 mg, 150 μl of phosphate buffered saline) was injected subcutaneously into the mid-cheek region of the face bilaterally using a 27-gauge needle. Additional animals (N=6) were inflamed in the same manner, but were administered morphine (s.c., 0.5 mg/kg, 200 μl) 2.5 hours post-carrageenan injection. All animals were then tested using the thermal operant device 3 hours post-inflammation at 45.5° C. and the six outcome measures (intake, licking events, facial contact events and duration, ratio licking/contact events, and ratio facial duration/facial contacts) were collected.

Statistical analysis. Data normality was assessed (Kolmogorov-Smimov with Lilliefors Significance test) and the appropriate statistical analyses were completed (ANOVA for repeated measures, or Kruskal-Wallis test) to determine whether the effects of temperature were significant. An ANOVA was used to evaluate significant treatment (none, inflammation, inflammation/morphine) effects at 45.5° C. on outcome measures. When significant differences were found, post-hoc comparisons were made using the Dunnett's test or the Mann-Whitney U test, using a probability level of 0.05.

It should be understood that the examples and embodiments described herein are for illustrative purposes only and that various modifications or changes in light thereof will be suggested to persons skilled in the art and are to be included within the spirit and purview of this application.

### Example 1

### Intake Threshold and Animal Training

Intake threshold was used to assess whether an animal had learned the operant reward task. In a preliminary experiment, a set of animals (N=10) tested at 24.3° C. had an intake of 10.95±1.21 g. Based on these data, a criterion of >10 g was set to consider an animal as trained. The average intake for the baseline sessions of this study was 11.01±1.10 g. Animal weight was monitored during the course of the study and was not significantly altered beyond normal weight gain.

### Example 2

### Assessment of the Effect of Testing Order

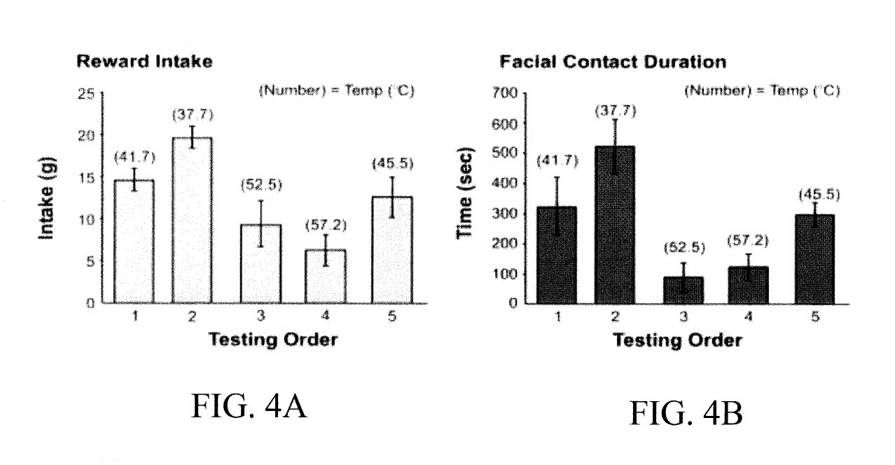

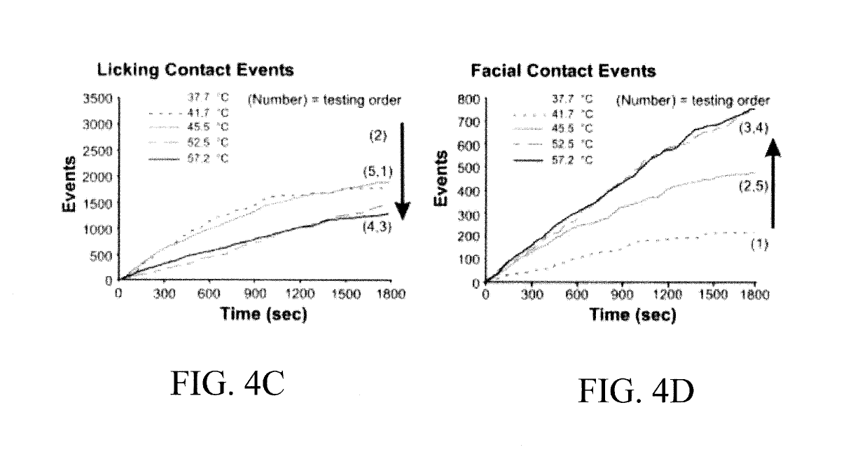

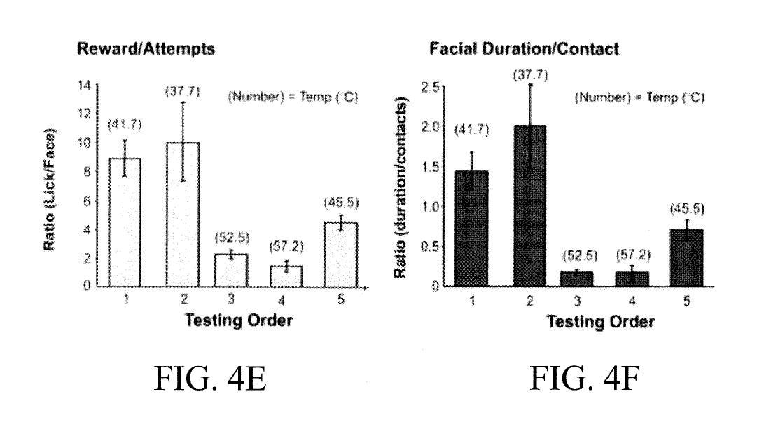

As seen in FIGS. 4A-4F, there was not an association of the outcome measures with the testing order, as each outcome did not increase or decrease according to the testing sequence. Based on the results of these animals, the outcome measures (intake, licking events, facial contact events and duration, ratio licking/contact events, and ratio facial duration/facial contacts) from the 52.5 and 57.5° C. testing sessions were compared and it was determined that these outcomes were not significantly different (Table 1). Additionally, these temperatures are both above the nociceptive threshold level and are likely activating the same subset of nociceptors. Thus these data were pooled (N=18) for subsequent analyses.

TABLE 1
Comparison of the 52.5 and 57.2° C.
testing sessions on outcome measures

| Outcome measure | 52.5° C. | 57.2° C. | Sig. |
|---|---|---|---|
| Intake | 8.3 ± 2.9 | 5.8 ± 1.7 | 0.959 |
| Licking contact events | 1492 ± 546 | 1290 ± 541 | 0.798 |
| Facial contact events | 761 ± 212 | 753 ± 390 | 0.505 |
| Facial contact duration | 123 ± 37 | 89 ± 42 | 0.574 |
| Ration of reward/attempts | 1.45 ± 0.36 | 2.32 ± 0.28 | 0.161 |
| Ratio of facial duration/contacts | 0.17 ± 0.03 | 0.19 ± 0.09 | 0.382 |

There were no significant differences for each of the facial testing outcome measures (mean±s.e.m.); therefore, data were pooled for subsequent analyses. (Sig.: calculated level of significance).

### Example 3

### Intake and Facial Contact Duration

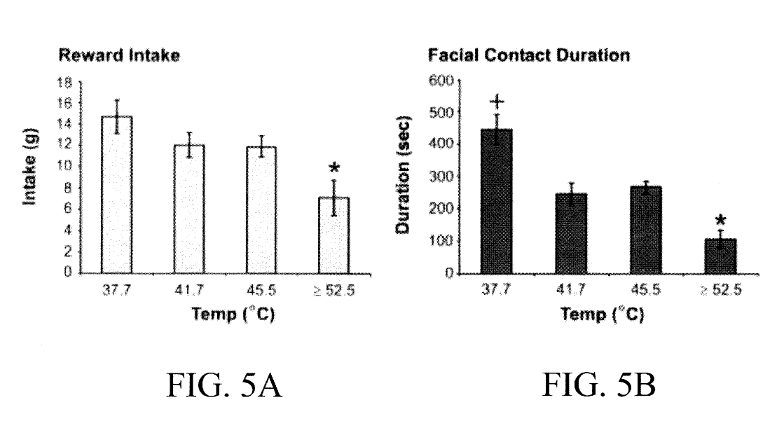

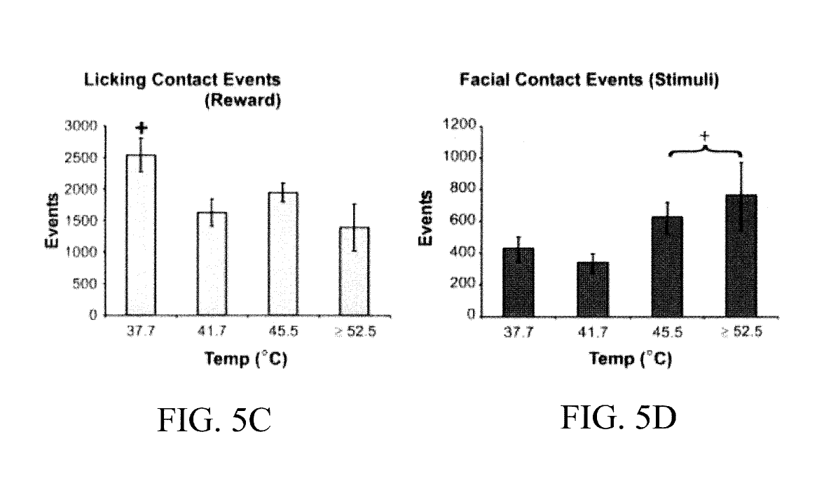

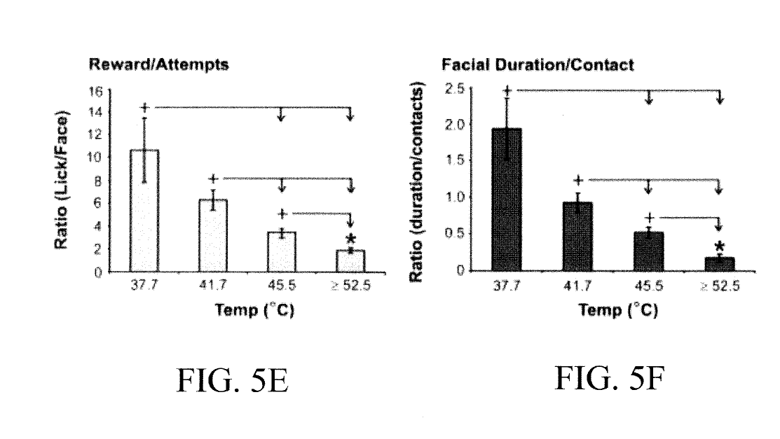

There was a significant effect of temperature on both reward solution intake (FIG. 5A, F=4.87, P<0.005) and total facial contact duration (FIG. 5B, F=16.79, P<0.0001). The highest testing temperatures (≧52.5° C.) produced a significantly lower reward solution intake and shorter total facial contact durations as compared to lower temperatures. Facial contact duration was significantly longer (P<0.05) in the 37.7° C. sessions as compared to the higher temperatures. No animals displayed swelling, blistering, redness or any indicators of tissue damage following any of the sessions.

### Example 4

### Licking and Facial Contacting Events

Two other operant measurements that were recorded online during the testing sessions were (1) contacts with the reward bottle (licking contact events) and (2) contacts with the stimulus thermode (facial contact events). Licking contact data were used to assess the success of the animal for obtaining the reward and facial contact data provided information regarding the attempts at the reward. As shown in FIG. 5C, there was a significant decrease (F=3.98, P<0.05) in the number of licking events as the stimulus temperature increased. The neutral temperature (37.7° C.) produced significantly more licking events as compared to all of the other temperatures tested. The number of facial contacts was also significantly affected by the temperature of the thermode (F=3.387, P<0.05). FIG. 5D illustrates that temperatures ≧45.5° C. resulted in a significantly larger number of facial contact events as compared with lower temperatures.

Two pain indices were derived from the licking and facial contact outcome measures. The first index (FIG. 5E), the ratio of Licks/Attempts compares the cumulative numbers of reward successes (Licking Contact Events) to the cumulative number of attempts (Facial Contact Events). There was a significant effect of stimulus temperature (F=7.24, P<0.01) on this Licks/Attempts ratio, and all response ratios were statistically lower (P<0.05) at successively higher temperatures with the exception of 37.7 and 41.7° C. The second index (FIG. 5F), compared the ratio of facial duration per facial contact and was also significantly affected by increasing stimulus temperature (F=12.35, P<0.0001). Again, all response ratios for the facial duration/contact were statistically lower (P<0.05) at successively higher temperatures with the exception of 37.7 and 41.7° C. These ratios provided another indication of aversion, as the animals were less inclined continue past the thermode to receive reward with increasing nociceptive stimulus intensity.

### Example 5

### Between and within Group Comparisons

There were no significant differences for the six outcome measures (intake, licking events, facial contact and duration, ratio licks/contact events, and ratio facial duration/facial contact) when a between group (N=8 vs. 10) analysis of testing sequence was completed at 37.7, 45.5 and ≧52.5° C. The between group comparison at 42° C. was also not significant for five of the six outcome measures (intake, licking events, facial contact and duration, and ratio licking/contact events), the exception being the duration/contact ratio comparison.

In order to assess the effect of testing experience, a within group (N=10) comparison using the 45.5° C. stimulus was completed. There were no significant differences between the two testing sessions at this temperature for each of the outcome measures.

### Example 6

### Effects of Orofacial Inflammatory Pain and Analgesia on Orofacial Operant Outcome Measures

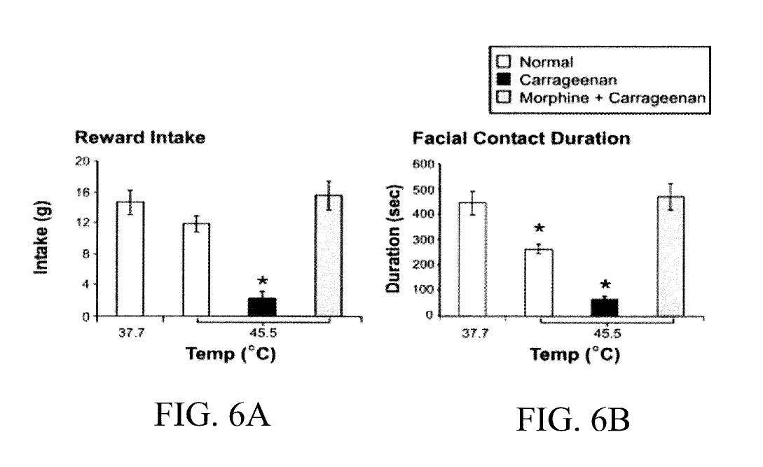

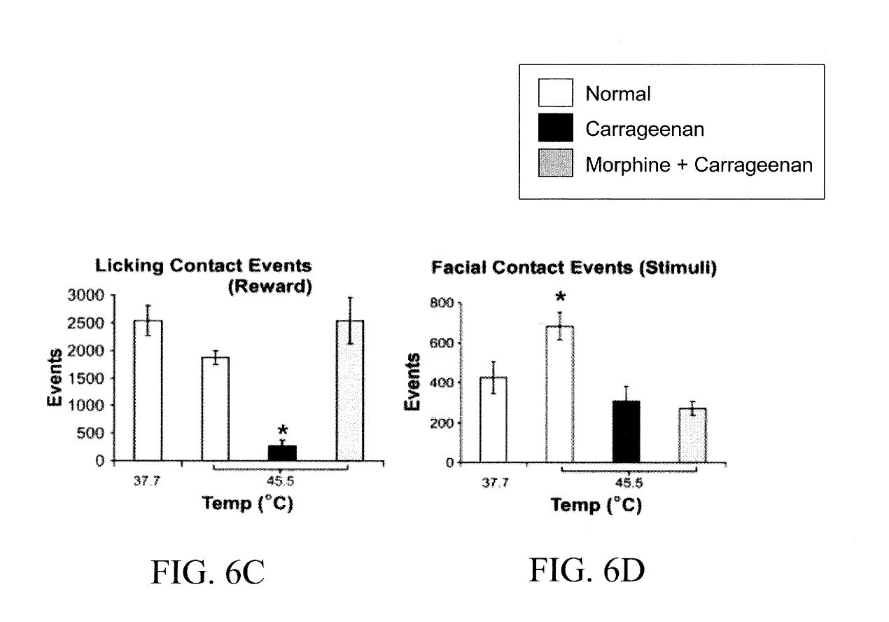

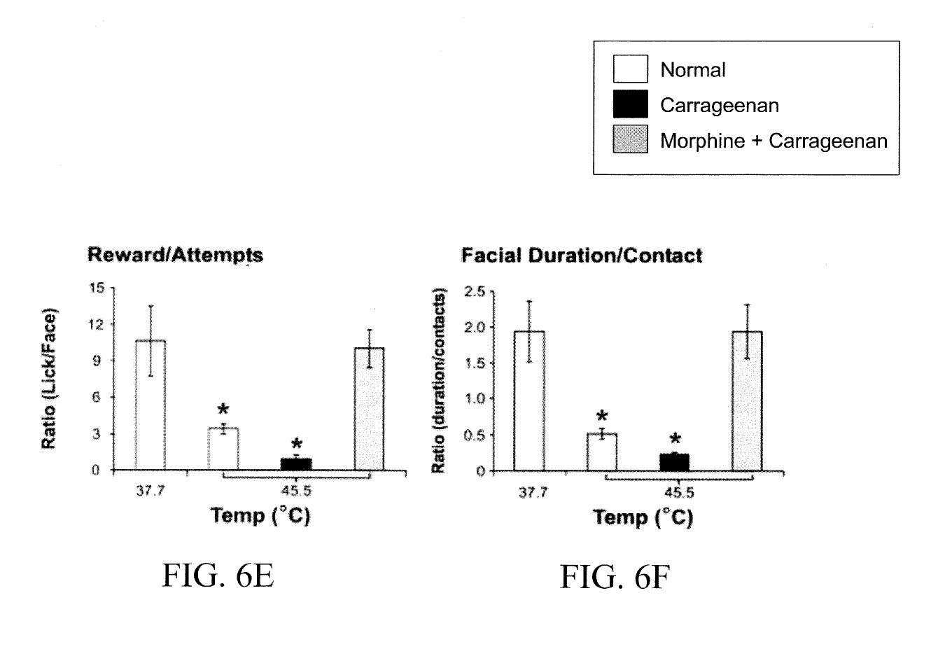

There were significant treatment effects (none, inflammation, inflammation/morphine) at 45.5° C. on all of the outcome measures: intake (F=10.89, P<0.001), licking contact events (F=15.31, P<0.001), facial contact events (F=5.72, P<0.005) and duration (F=23.07, P<0.001), ratio licks/contact events (F=26.21, P<0.001), and the ratio facial duration/facial contacts (F=5.56, P<0.001). Inflammation produced a significant decrease in all six of the outcome measures and all but one of these effects was completely reversed when animals were pre-treated 30 minutes prior to testing with morphine (FIGS. 6A-6F). All operant outcome measures except facial contact events for the morphine-treated animals tested at 45.5° C. were comparable to normal animals tested at 37.7° C., indicating an inhibition of both the inflammatory hyperalgesia and normal thermal pain produced at 45.5° C.

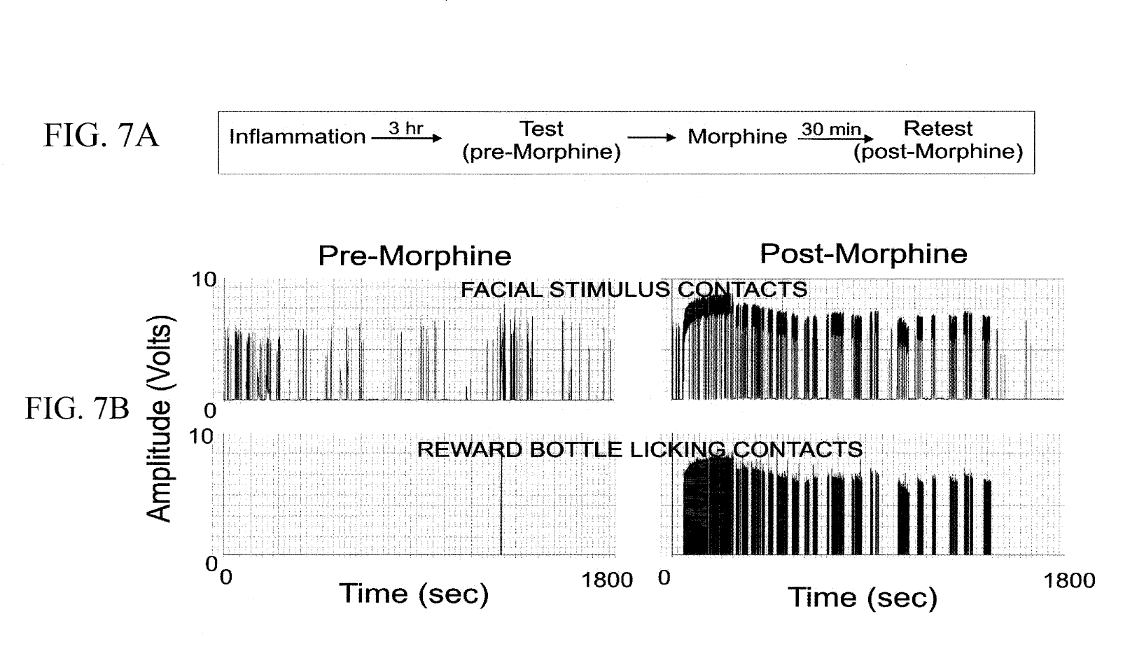

To further illustrate the effects of morphine on inflammatory pain, one animal was tested 3 hours post-inflammation using the standard 30-minute operant trial and then this animal was given morphine (0.5 mg/kg, s.c.) and retested 30 minutes later (FIGS. 7A-7B). There was a reversal of the outcome measures following morphine administration in the presence of inflammation (values given as pre- vs. post-morphine): intake (1.12 vs. 17.8 g), licking events (19 vs. 3582), facial contacts (437 vs. 309), duration (94 vs. 634 s), ratio licking/contact events (0.04 vs. 11.6), and ratio facial duration/facial contact (0.21 vs. 2.05).

Pain evaluation in animal models typically involves assessment of: segmental withdrawal reflexes, more complex unlearned (innate) behaviors, or learned operant behaviors (Chapman, C. R. et al. Pain, 1985, 22:1-31). While no one behavioral assay evaluates the full spectrum of nociceptive responses, there are distinct advantages and limitations for each assay. Segmental reflexes involve links between sensory inputs and motoneurons and modulation of the spinal reflex circuit via local interneurons and descending tracts. For example, tail-flick reflexes can be elicited by aiming a heat source onto an animal's tail to produce withdrawal responses (D'Amour, F. E. and Smith, D. L. J. Pharmacol Exp Ther, 1941, 72:74). This reflex requires processing within the spinal cord and is under supraspinal control. Advantages of simple reflex assays include their relative ease, and the results can be related to human studies of similar responses. A limitation, of reflex testing is that outcomes do not necessarily measure pain, but rather provide measures of sensorimotor integration at the segmental level (Chapman, C. R. et al. Pain, 1985, 22:1-31). For example, the tail-reflex and limb withdrawal responses can be elicited in spinalized animals (Kauppila, T. et al. Brain Res, 1998, 797:234-242). Additionally, if motor function is compromised, then these outcomes can be affected, which is important when considering reflex attenuation.

Complex, unlearned behaviors are mediated by brain stem processing and include paw licking, face-rubbing, limb guarding, vocalization, grooming, chewing/biting, or a combination of these behaviors (Benoliel, R. et al. Pain, 2002, 99:567-578; Berridge, K. C. Behav Brain Res, 1989, 33:241-253; Hartwig, A. C. et al. J. Oral Maxillofac Surg, 2003, 61:1302-1309; Hargreaves, K. et al. Pain, 1988, 32:77-88; Kayser, V. and Guilbaud, G. Pain, 1987, 28:99-107; Rosenfeld, J. P. et al. Pain, 1983, 15:145-155; and van Eick, A. J. Acta Physiol Pharmacol Neerl, 1967, 14:499-500). For example, in the hot plate test, one can monitor a variety of behaviors following stimulation, including licking and guarding (van Eick, A. J. Acta Physiol Pharmacol Neerl, 1967, 14:499-500). These unlearned behaviors can be present in decerebrate animals (Woolf, C. J. Pain, 1984, 18:325-343).

Although simple reflexes and other unlearned behaviors are easily evaluated for the hindpaw or tail, assessment of orofacial pain in animals has been more challenging. A few methods have been utilized for the face such as recording withdrawal responses to mechanical stimuli or heat or observing innate behaviors such as grooming (Clavelou, P. et al. Pain, 1995, 62:295-301; Vos, B. P. et al. J. Neurosci., 1994, 14:2708-2723). However, these approaches do not provide information about higher order cerebral processing and provide only partial information relating to trigeminal nociceptive modulation. Operant responses involve complex behavioral actions and are advantageous in that the animal has control over the amount of nociceptive stimulation and can modify its behavior based on cerebral processing (Mauderli, A. P. et al. J. Neurosci Methods, 2000, 97:19-29; Vierck Jr, C. J. et al. Neuroscience, 2003, 119:223-232). Conflict paradigms involve learned operant behaviors that reflect animals' choices between receiving a positive reward or escaping aversive stimuli (Dubner, R. et al. “A behavioral animal model for the study of pain mechanisms in primates” in Weisenberg, M. Tursky, B. Eds., Pain: New Perspectives in Therapy and Research, New York: Plenum Press, 1976, pp. 155-170; Vierck Jr., C. J. et al. Exp Brain Res, 1971, 13:140-158).

Previous studies have evaluated the influence of the laboratory environment on animal behaviors and found that different experimenters can be an important influence on behavioral outcome measures (Chesler, E. J. et al. Neurosci Biobehav Rev, 2002, 26:907-923; Crabbe, J. C. et al. Science, 1999, 284:1670-1672). Therefore, development of investigator-independent measures becomes especially relevant when evaluating pain behaviors. Data achieved via operant testing are typically not experimenter derived or driven. This is important when one considers testing in the orofacial region, as visual cues or restraints may contribute major confounding factors (e.g., stress). Additionally, if an experimental manipulation produces a motor disability, control procedures for an operant behavioral assay that incorporate the same motor response are capable of demonstrating this behavioral change.

Currently, there are few operant models for assessing orofacial pain in rodents. Harper et al. successfully evaluated specific aspects of feeding behavior in a model of temporomandibular joint (TMJ) pain (Harper, R. P. et al. J. Dent Res, 2000, 79:1704-1711). They demonstrated that inflammation of the TMJ produced significant changes in food intake and meal patterns for rats. These outcomes may be considered a form of operant conditioning, as the animal must choose to either eat with pain or not eat. While these outcomes provided a non-invasive means of demonstrating and quantifying TMJ pain, they did not discriminate between specific components of pain (i.e., hyperalgesia/allodynia). The conflict paradigm described herein expands on this previous work and lends itself to adaptation to the facial region, e.g., the orofacial region. This approach is advantageous in that the animal is able to gradate its level of participation. This was readily apparent once the stimulus temperature was adjusted into a nociceptive and potentially tissue damaging range in which none of the animals suffered obvious tissue damage (i.e., blistering, redness, and swelling).

Reward intake at the highest stimulus temperatures (≧52.5° C.) was significantly decreased; however, intake did not discriminate between the neutral, warm and hot temperatures (37.7-45.5° C.). There was a significant decrease in the amount of time an animal placed its face on the thermode at the highest temperatures, but this measure did not discriminate between two intermediate temperatures (41.7, 45.5° C.). Thus, intake and cumulative stimulus duration do not appear to be sensitive enough measures for discriminating between warm and hot temperatures, demonstrating that the reward conflict, as expected, produces a degree of pain tolerance.

When evaluating licking contact events, again there did not appear to be a distinction between responses in the warm to hot range. However, the animals adapted a technique at the higher temperatures where they would dart on and off the thermode, attempting to quickly lick the bottle. These animals attempted to compensate for short drinking periods, each terminated by escape from nociceptive stimulation, by increasing their frequency of attempts; however, they were less successful at drinking the reward solution at the high temperatures. There was an increase in the number of facial contact attempts and a decrease in successful licking contact events as the stimulus increased from neutral/warm to hot. Thus, the facial contact events may be interpreted a variety of ways. For example, a reduced number of stimulus contact events can denote either increased or decreased pain. In the former instance, aversion to the stimulus could be sufficiently strong to discourage contacts with the thermode. Conversely, an analgesic can be expected to produce a low number of facial contacts with longer stimulus-contact duration.

In order to resolve this paradox and distinguish painful from non-painful states, two ratios derived from the aforementioned outcome measures were evaluated. A significant stimulus-dependent decrease in the ratio of reward/attempts with increasing temperature was observed; therefore, a low ratio was considered representative of a painful behavior. Contact with the nociceptive stimulus can prevent access to the reward and/or the animal can adopt a strategy whereby acquisition of positive reward involves more attempts, albeit at shorter duration, as the stimulus reaches a nociceptive level. Conversely, a high number of reward access events coupled with a low number of stimuli contact events, produces a high ratio, indicative of a minimal or non-painful response to thermode contact.

The stimulus duration/contact event ratio provided another discriminating variable. For example, a low number of stimulus contacts coupled with a long duration would be an indication of a non-painful state, thus producing high ratio values. Low ratios from a low contact number and corresponding short contact duration would be indicative of a painful state. These two ratios provide sensitive measures for detecting behavioral changes produced by small changes in temperature and allow for discrimination of responses to neutral, warm and hot temperatures. Under inflammatory conditions, these ratios were significantly reduced at 45.5° C., indicating a hyperalgesic response. This hyperalgesia was completely reversed following administration of a clinically relevant dose of morphine. These ratios will be critical for future studies involving other orofacial pain models in regards to characterizing development of hyperalgesia versus allodynia under pathological conditions.

Limitations of operant-derived assays include the necessity to use unique devices that may not be readily available; therefore replication of results from other investigators becomes a relevant issue. Additionally, baseline training is required, as the animal must learn an adaptive behavior. The operant orofacial device described herein is both simple to construct and operate, thus it overcomes challenges that can be associated with operant tests. Baseline training of this orofacial testing device is minimal, requiring just a few sessions, and data collection is automated, allowing for high data throughput collection.

In summary, the present inventors demonstrate that an orofacial pain behavior profile can be assessed based on an animal's response in a conflict testing environment. A critically important feature of algesiometric testing is that it reveal stimulus-response relationships appropriately related to nociceptive processing (Vierck Jr, C. J. et al. Neuroscience, 2003, 119:223-232), and this criterion was met by the method described here. These data help fill a current void in animal orofacial pain assessment by providing behavioral measures that express physiological and cerebral processing of pain, thus allowing for evaluation of a number of factors relating to pain coding. This operant orofacial pain assessment system can be used to evaluate behavior in a variety of pain models. The present inventors anticipate that this system will play an important role in distinguishing heat hyperalgesia from heat allodynia by providing a stimulus-response function for stimuli that normally do and do not elicit aversion. This simple, yet versatile system will allow for screening of therapeutics aimed at treating facial pain such as orofacial pain.

### Example 7

### Mechanical Aversive Stimulus

This embodiment of the device includes grounded metal filaments adjacent and/or within the hole. In this example, the chamber is a clear acrylic box through which the animal must place its head through a hole to drink a reward solution (e.g., sweetened condensed milk) from a standard rodent watering bottle. Preferably, the metal filaments are removable such that they can be interchanged with filaments of various diameters and/or mechanical resistance. The metal spout on the watering bottle will be connected to a DC power supply and, in series, to a computer data acquisition system. The bottle will be placed in a position such that the animal, when reaching towards it with its face, will contact the filaments. The bottle position can be adjusted horizontally and vertically to target specific areas of the facial region (i.e., maxillary or mandibular divisions). When the animal drinks from the water bottle the skin on its' shaved face will contact the grounded metal filaments and the animal's tongue will contact the metal spout on the water bottle, completing the electrical circuit (registered in the computer as a 3 volt step). The frequency of drinking, the total number of events, the duration of the drinking events, and the time course of the drinking will be recorded. Additionally, the total amount of milk consumed can be measured and compared between animals and treatments. A similar circuit will be established from the metal filaments to the animal by grounding the floor with an aluminum sheet, and the same parameters will be recorded to identify to determine the frequency of contacting the filaments, the total number of events, and the duration of the contacts. This circuit is necessary to determine if the animal makes an attempt at the condensed milk but is discouraged by the mechanical stimulation of the filaments. A complete session will typically last 30 minutes per trial and animals can be tested 3 or 4 times a week and will be fasted prior to each testing session.

The present inventors expect that the use of this innovative operant behavioral assay will yield a novel and sensitive means of detecting and quantifying pain within the facial region and can be completed on several models of facial pain (e.g., inflammation, arthritis, nerve injury). The usefulness of this device derives from the simplicity of the design and the wealth of data that can be generated. Typical pain assays evaluate rudimentary segments of the pain processing pathway, such as reflex (e.g., limb withdrawal) or unlearned behaviors (e.g., grooming). However, in order to better model the human pain experience, one must consider the effects of higher processing done in the brain. In this reward-conflict assay, this higher level of processing can be assessed whereby the animal must make its own decision on whether it will complete the task based on its pain level. This closer simulates human pain conditions as motivation and emotional states influence the experience. This method would be provide an automated operant assay for evaluating mechanical sensitivity associated with pain within the facial region and provides a pivotal link for translating basic pain research ideas into clinic trial strategies for managing pain.

This non-invasive thermal assessment assay provides a way of assessing both mechanical hyperalgesia and allodynia. This type of assessment is novel due to the fact that there are currently no automated, operant behavioral devices available that are capable of evaluating these outcome measures in the facial region. Additionally, since the animals are unrestrained, there are less confounding factors such as stress, which are inherent to other facial pain testing techniques. A significant benefit of this device is that the procedure can be automated once the animal is placed in the box, therefore, a high throughput for behavioral data can be obtained.

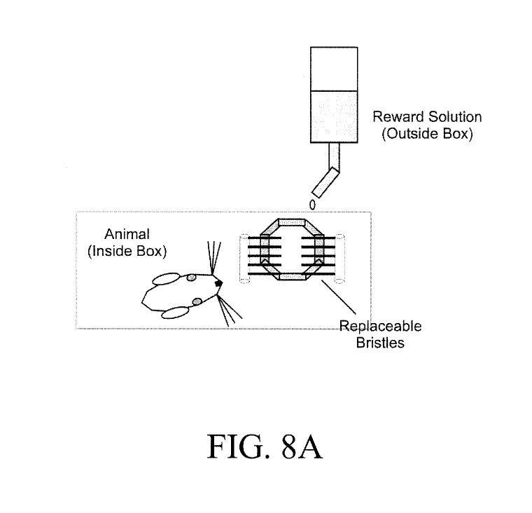

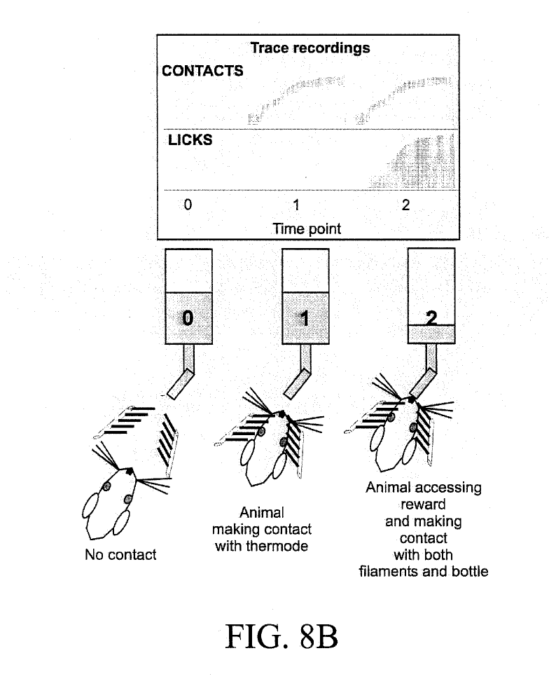

In those embodiments of the device that utilize bristles as a means for producing an aversive mechanical stimulus (as shown in FIGS. 8A and 8B), the bristles may be of any length, orientation, or composition (e.g., metallic, polymeric, natural or manmade fibers/filaments) sufficient to provide an aversive mechanical stimulus to the animal without injury.

All patents, patent applications, provisional applications, and publications referred to or cited herein are incorporated by reference in their entirety, including all figures and tables, to the extent they are not inconsistent with the explicit teachings of this specification.

1-20. (canceled)

21. A device comprising:
a) a means for providing an aversive stimulus to a test animal; and
b) a means for providing a reward to the test animal, wherein said means for providing the aversive stimulus and said means for providing the reward are spatially arranged with respect to one another such that the test animal must move into contact with the means for providing an aversive stimulus in order to access the reward;
wherein the aversive stimulus is an aversive temperature or mechanical stimulus; and
wherein the test animal's face must contact the means for providing the aversive stimulus in order to access the reward; and
wherein the device is configured to follow a reward/conflict paradigm.

22-24. (canceled)

25. The device of claim 21, wherein an electrical circuit is completed when the test animal contacts both the means for providing the aversive stimulus and the means for providing the reward.

26. The device of claim 21, wherein the aversive stimulus is an aversive temperature.

27. The device of claim 26, wherein said means for providing the aversive stimulus comprises a thermode.

28-29. (canceled)

30. The device of claim 21, wherein said means for providing the aversive stimulus and said means for providing the reward are spatially arranged with respect to one another such that the test animal must be in contact with the means for providing the aversive stimulus while accessing the reward.

31. The device of claim 21, further comprising a chamber, wherein said chamber has a wall with an aperture through which the test animal must extend its head to access the reward.

32. The device of claim 31, wherein said means for providing the aversive stimulus is outside said chamber and adjacent to said aperture or within said chamber and adjacent to said aperture.

33. The device of claim 31, wherein said means for providing the aversive stimulus at least partially lines said aperture.

34. The device of claim 21, further comprising a partition enclosing said means for providing the reward, wherein the partition has an aperture through which the test animal must extend its head to access the reward.

35. The device of claim 21, further comprising a chamber, wherein said means for providing the aversive stimulus, said means for providing the reward, and a said partition are located within said chamber.

36. The device of claim 21, wherein the aversive stimulus is mechanical stimulus.

37. The device of claim 36, wherein said means for providing the aversive mechanical stimulus comprises one or more filaments.

38. The device of claim 21, wherein the device comprises two of said means for providing aversive stimuli, wherein a first means for providing aversive stimulus provides aversive mechanical stimulus and wherein a second means for providing aversive stimulus provides aversive temperature stimulus.

39. The device of claim 21, wherein the device comprises two of said means for providing aversive stimuli, wherein a first means for providing aversive stimulus provides aversive cold temperature and wherein a second means for providing aversive stimulus provides aversive hot temperature.

40. The device of claim 21, further comprising a computer data acquisition system, wherein said computer data acquisition system is in operable communication with said means for providing the aversive stimulus, or with said means for providing the reward, or with both.

41. A method for testing pain sensitivity exhibited by a test animal, comprising introducing the test animal to a device; and determining at least one pain measure from the test animal, wherein the device comprises:
a) a means for providing an aversive stimulus to a test animal; and
b) a means for providing a reward to the test animal, wherein said means for providing the aversive stimulus and said means for providing the reward are spatially arranged with respect to one another such that the test animal must move into contact with the means for providing an aversive stimulus in order to access the reward;
wherein the aversive stimulus is an aversive temperature or mechanical stimulus; and
wherein the at least one pain measure is a measure of orofacial pain of the test animal; and
wherein said means for providing the aversive stimulus and said means for providing the reward are spatially arranged with respect to one another such that the test animal must be in contact with the means for providing the aversive stimulus while accessing the reward; and wherein the device is configured to follow a reward/conflict paradigm.

42. The method of claim 41, wherein the at least one pain measure includes one or more selected from the group consisting of: the number of times the test animal accesses the reward; the amount of reward taken; the number of times the test animal contacts the means for providing the aversive stimulus; the ratio of the number of times the test animal accesses the reward to the number of times the test animal contacts the means for providing the aversive stimulus; and the relationship between the duration of contact between the test animal and the means for providing the aversive stimulus and the number of times the test animal contacts the means for providing the aversive stimulus.

43. The method of claim 41, further comprising subjecting the test animal to a treatment before, during, or after determining at least one pain measure from the test animal.

44. The device of claim 21, wherein the device is configured such that, when in use, the test animal is not restrained.

45. The method of claim 41, wherein introducing the test animal to the device comprises introducing the test animal unrestrained to the device; and
wherein the step of determining at least one pain measure from the test animal is performed while the test animal is unrestrained.
